(19) United States
(12) Patent Application Publication
(10) Pub. No.: US 2017/0367563 A1
(43) Pub. Date: Dec. 28, 2017

(54) AIRBAG TUBE APPARATUS AND METHOD OF OPERATION

(71) Applicants: Chung-Yung HO, Taipei (TW); Chung-Chen HO, Taipei (TW); Hong-Fa HO, Taipei (TW)

(72) Inventors: Chung-Yung HO, Taipei (TW); Chung-Chen HO, Taipei (TW); Hong-Fa HO, Taipei (TW)

(21) Appl. No.: 15/630,213

(22) Filed: Jun. 22, 2017

(30) Foreign Application Priority Data
Jun. 23, 2016 (TW) 105119763

(51) Int. Cl. A61B 1/00; A61M 25/10; A61B 1/06; A61B 1/05; A61B 1/045; A61F 5/451

(52) U.S. Cl. CPC A61B 1/00082; A61B 1/045; A61F 5/451; A61B 1/0676; A61B 1/05; A61M 25/10185; A61B 1/00195; A61B 1/00045; A61B 1/00006; A61M 2205/50

(57) ABSTRACT

An airbag tube apparatus has a hollow tube with a closed end and an open end. A side hole is disposed near the closed end; when matter leaves the airbag tube apparatus, it enters the hollow tube from the side hole and flows out of the open end. A balloon is attached to the hollow tube, and connected to a vent. The balloon in a large volume can flexibly fix animal tissue. A vent is connected to the balloon, a valve, and a pneumatic module to form an air inflator to inflate, hold and deflate the balloon. The valve opens and closes the liquid flow in the vent, and is controlled by a circuit module; the pneumatic module, providing inflating function, is controlled by the circuit module; the circuit module, has a microprocessor unit, a memory unit and an input-and-output unit.

### FIELD OF THE INVENTION

The present invention relates to medical apparatus field, particularly to an airbag tube apparatus and an operation method thereof.

### BACKGROUND OF THE INVENTION

Medical devices contains varies airbag tubes and the operation method thereof. For example, airbag catheters, airbag faeces drainage tube, airbag tracheaostomy tube, endotracheal tube, airbag oropharynx tracheal tube, etc and the operation method thereof. An airbag anal drainage bag disclosed in the Chinese patent database with publishing number CN202051874U, and a four-chamber double-airbag catheter disclosed in the Chinese patent database with publishing number CN103463727A are airbag tube apparatus to drain urine and faeces.

These apparatus has disadvantages after practical, for example, the balloon has to deflate by manual work every once in a while to prevent the problems of ischemic necrosis of the tissue due to overlong inflecting period of balloon, leakage of the balloon, discomfort of the patient feeling the balloon pressing the tissue, the apparatus piecing or damaging the tissue as the operator is unable to view the situation, the operator unaware of the airbag tube sliding out. Above problems are needed to overcome.

### SUMMARY OF THE INVENTION

The present invention is provided that a circuit module and a pneumatic module are configured to achieve automatically inflating and deflating of the balloons to overcome the problem of ischemic necrosis of the tissue (like intestine wall) due to overlong inflecting period of balloon; the balloon can be automatically adjusted of the pressure in the balloon is decreased or is overhigh; if the patient feels uncomfortable, the balloon can be automatically adjusted according to preset parameter; endoscopy and ultrasonic module are configured to prevent the apparatus piercing or damaging the tissue due to unvisual of the situations in the path of the apparatus; communication function is configured to provide remote information and remote control; detecting module is configured to detect that whether the airbag tube slides out. The present invention further provides the operating method of the apparatus. The present invention is different from the existing known technology. The airbag of the present invention comprises gases and fluids.

An airbag tube apparatus of the present invention comprises: a hollow tube 11 with a closed end 19 and an open end 20; a side hole 18 is disposed near the closed end 19; when the apparatus of the present invention is used to drain some matter, the matter enters the hollow pipe 11 from the side hole 18 and then flows out of the open end 20; a balloon 12, which is attached to the hollow pipe 11, is connected to a vent 13, the balloon shrinks when no liquid (gases or fluids) exists in the balloon and becomes larger when liquid fills the balloon, the balloon in a large volume can lock animal tissue, making the apparatus flexibly fixed to the animal tissue; a vent 13 is connected to the balloon 12, a valve 14, a pneumatic module 15 to form an air inflator to inflate, hold and deflate the balloon 12; the valve 14, used to open and close the liquid flow in the vent 13, is controlled by a circuit module 16; the pneumatic module 15, providing inflating function, is controlled by the circuit module 16; the circuit module 16, comprising a microprocessor unit, a memory unit and an input-and-output unit, provides default parameters, amended parameters and count-down functions and controls the open and close of the valve 14 and the inflating of the pneumatic module 15; a power supply 17 provides power to the apparatus; the balloon 12 is inflated, held and deflated automatically in accord with a preset period.

An operating method of the airbag tube apparatus comprises the steps: (1a) the operator determines that the balloon 12 is deflated completely, the balloon shrinks until the apparatus can enter the animal tissue; (1b) the operator puts the airbag tube 10 to a target position, like rectum or urethra of a human body; (1c) the circuit module 16 controls the valve 14 to make gases in the balloon 12 not leak out, and starts up the pneumatic module 15 to inflate the balloon 12 until the gas volume of the balloon 12 reaches the preset parameter, the balloon has the volume increased to achieve that the apparatus of the present invention can flexibly fix to animal tissue; (1d) the circuit module 16 counts down (according to the preset inflating maintaining period), the valve 14 is changed at the end of the period, the gases in the balloon 12 leaks out to deflate, the balloon 12 shrinks that the apparatus can not flexibly fix to the animal tissue; (1e) the circuit module 16 counts down according to preset deflating maintaining period, performing step (1c) at the end of the period, the balloon 12 is inflated, held and deflated automatically in accord with a preset period. The present invention further comprises at least one balloon 12, at least one vent 13, at least one valve 14 and at last one side hole 8; the circuit module 16 controls the pneumatic module 15 and the valve 14 to inflate, hold and deflate the balloons 12. The balloons are changed to press the tissue area, preventing the problem of ischemic necrosis of the tissue like intestine wall due to overlong inflecting period of balloon.

The present invention is further provided that the hollow tube 11 is disposed with a transparent area, a camera module 22, an illumination module 21 and a display module 24; the camera module 22 comprises necessary power wires and signal wires. The camera module 22 moves in and out of the hollow tube 11; the transparent area of the hollow tube 11 is provided for the illumination module 21 to illuminate of and for the camera module 22 to shoot the periphery of the hollow tube 11; the transparent area of the hollow tube 11 can be a partial area or the whole area, for example, if the closed end 19 is transparent, images are transmitted to the display module 24, the operator can view the images from the display module 24, the display module 24 is a screen, a tablet computer, a computer or a hand phone; the illumination module 21 is a visual light resource, an infrared resource or any other light resource with any wavelength; the camera module 22 is configured with a panoramic lens, a non-panoramic lens, a wide-angle lens, a non-wide-angle lens, a fish-eye lens or other camera lens; the camera module 22 further comprises a ultrasonic transmitting and receiving unit. The operator can view the situations in the path of the apparatus in the animal body with the help of the illumination, the camera and the screen, preventing the apparatus piercing or damaging the tissue.

The present invention further provides an operating method of the airbag tube apparatus with camera module, illumination module and display module, the method comprises the steps: (2a) the operator determines that the balloon 12 is deflated completely, the camera module 22 and the illumination module 21 are put in the transparent area of the hollow tube 11, and the images of the camera module 22 displays in the display module 24; (2b) the operator views the images of the display module 24, and moves the airbag tube 10 to the target area; after the position of the airbag tube 10 is determines, to prevent the apparatus piercing or damaging the tissue, the operator moves the camera module 22, the illumination module 21 and the display module 24 out of the hollow tube 11, making the camera module 22, the illumination module 21 and the display module 24 not contacted with the hollow tube 11. At this time, a drainage bag 40 can be provided to connect to the open end 20 of the hollow tube 11 for drainage; (2c) the circuit module 16 controls the valve 14 to maintain the air of the balloon 12, the pneumatic module 15 inflates until the air volume of the balloon 12 reaches the preset parameter, the balloon has the volume increased to make the apparatus flexibly fix to the animal tissue. (2d) the circuit module 16 counts down according to the maintain period; the circuit module 16 changes the valve 14 at the end of the maintain period and deflates the balloon 12; the balloon shrinks that the apparatus can not flexibly fix to the animal tissue; (2e) the circuit module 16 counts down according to the deflation period; the circuit module 16 performs step (2c) at the end of the deflation period; the operator can watch the video the camera module 22 films during the airbag tube 10 moves to the target area; the balloon 12 is inflated, held and deflated automatically in accord with a preset period. The operator can view the situations in the path of the apparatus in the animal body, preventing the apparatus piercing or damaging the tissue. The apparatus of the present invention further comprises a detecting module 25 attached to the hollow tube 11, the detecting module 25 is electrically connected to the circuit module 16 to provide detecting value, like resistance value, capacitance value, inducting value, PH, etc, to the circuit module 16. The circuit module 16 judges when there is a change and determines a preset situation, it sounds out or alarms to automatically detecting that whether the airbag tube slides out of the animal body.

The present invention is further provided that the circuit module 16 is configured with communication function to communicate with a remote device 30 to provide the present information of the airbag tube 10 for long-distance operators or to receive parameters amended by long-distance operators through the remote device 30. The communication function is selected from WiFi, internet, Bluetooth, wire communication, wireless communication, optical communication; the remote device 30 is selected from computer, hand phone and tablet computer. This provides long-distance operators more detail information and more convenient operation. Each module and component have function to automatically maintain the air pressure of the balloon 12 according to the preset parameters, achieving automatically processing leaking problem of the balloon.

The present invention further comprises a steering unit 23 of the camera module 22 with mechanism and function to control the direction of the camera module 22 and the closed end 19 of the hollow tube 11. This mechanism and function is similar to a controllable camera of endoscopic or the same at the front end of the endoscopic. The present invention further comprises a drainage bag 40, the open end 20 of the hollow tube 11 is connected to the drainage bag 40, the drainage bag 40 is used to receive the drainage, the drainage bag 40 is configured with an outlet or without an outlet. When the apparatus is used to drain some matter, the drainage bag is used to store the matter like urine and faeces.

The method of the present invention further comprises a step that when the airbag tube 10 is put to the target area, the operator can operate the steering unit 23 of the camera module 22 to control the direction of the camera module 22 and the closed end 19 of the hollow tube 11. The operator can view the situations in the path of the apparatus in the animal body, preventing the apparatus piercing or damaging the tissue.

### BRIEF DESCRIPTION OF THE DRAWINGS

The present invention will be further described in accord with the drawings and the embodiments.

FIG. 1 illustrates a partial sectional and schematic diagram of the present invention.

FIG. 2 illustrates a flow diagram of the camera module, the illumination module and the display module of the present invention.

FIG. 3 illustrates a sectional diagram and schematic diagram of the present invention with a detecting module, a remote device and a drainage bag.

FIG. 4 illustrates a flow diagram of the present invention without a camera module.

FIG. 5 illustrates a flow diagram of the present invention with a camera module.

### REFERENCE SIGNS

Airbag tube 10
Hollow tube 11
Balloon 12
Vent 13
Valve 14
Pneumatic module 15
Circuit module 16
Power supply 17
Side hole 18
Closed end 19
Open end 20
Illumination module 21
Camera module 22
Steering unit 23
Display module 24
Detecting module 25
Remote device 30
Drainage bag 40

### DETAILED DESCRIPTION OF THE EMBODIMENTS

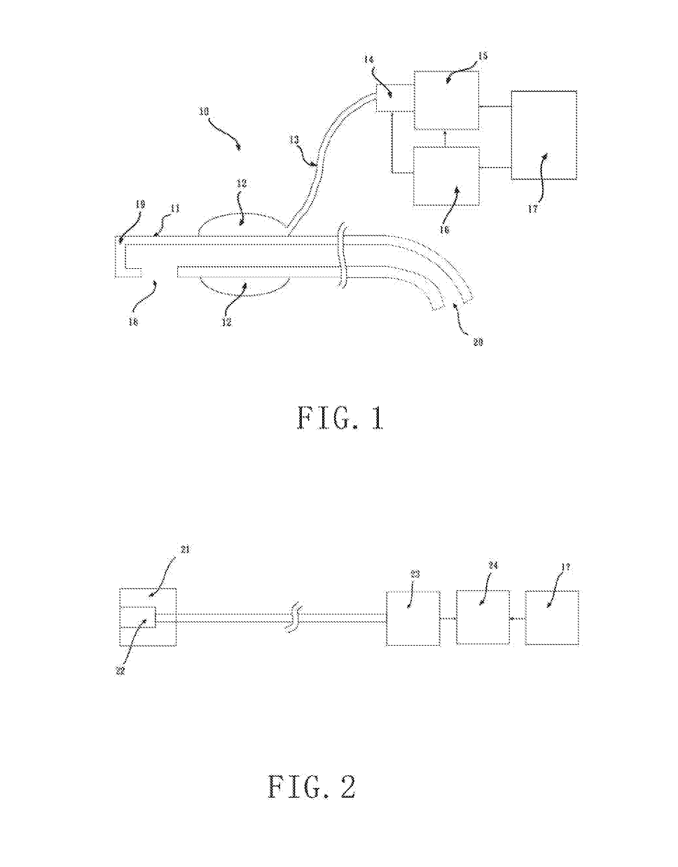

The present invention will be further described in accord with the embodiments. Please referring to FIG. 1, an airbag tube apparatus of the present invention comprises: a hollow tube 11 with a closed end 19 and an open end 20; a side hole 18 is disposed near the closed end 19; when the apparatus of the present invention is used to drain some matter, the matter enters the hollow pipe 11 from the side hole 18 and then flows out of the open end 20; a balloon 12, which is attached to the hollow pipe 11, is connected to a vent 13, the balloon shrinks when no liquid (gases or fluids) exists in the balloon and becomes larger when liquid fills the balloon, the balloon in a large volume can lock animal tissue, making the apparatus flexibly fixed to the animal tissue; a vent 13 is connected to the balloon 12, a valve 14, a pneumatic module 15 to form an air inflator to inflate, hold and deflate the balloon 12; the valve 14, used to open and close the liquid flow in the vent 13, is controlled by a circuit module 16; the pneumatic module 15, providing inflating function, is controlled by the circuit module 16; the circuit module 16, comprising a microprocessor unit, a memory unit and an input-and-output unit, provides default parameters, amended parameters and count-down functions and controls the open and close of the valve 14 and the inflating of the pneumatic module 15; a power supply 17 provides power to the apparatus; the balloon 12 is inflated, held and deflated automatically in accord with a preset period.

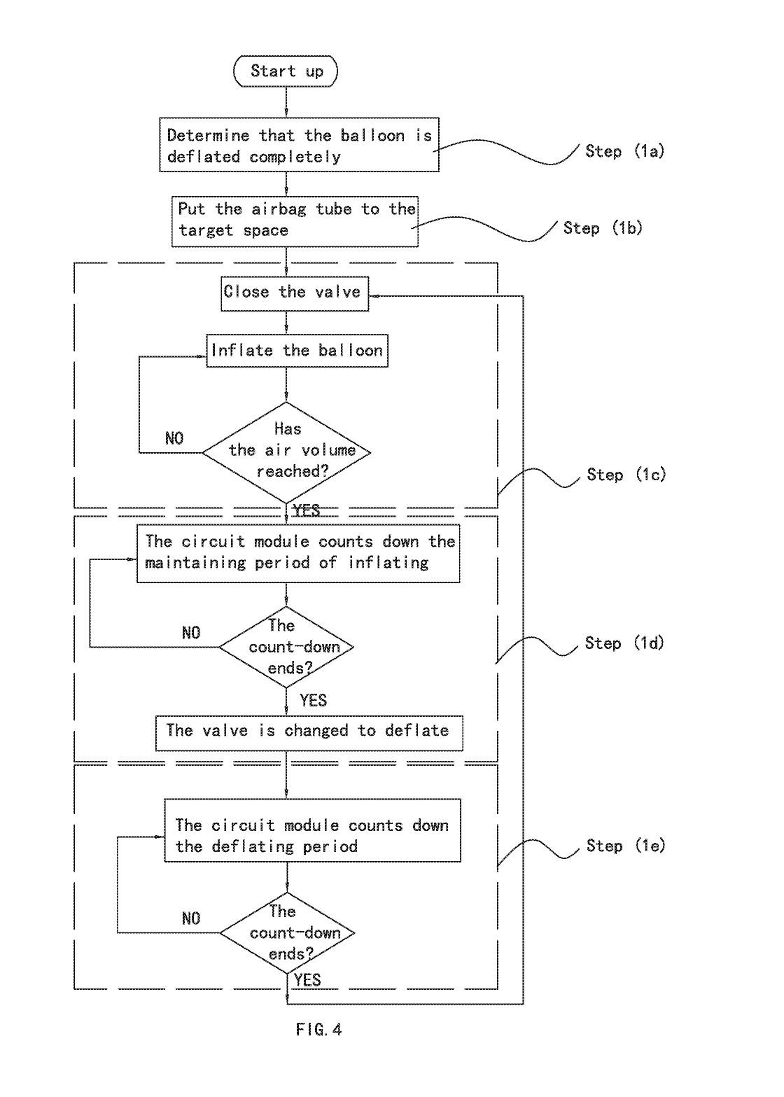

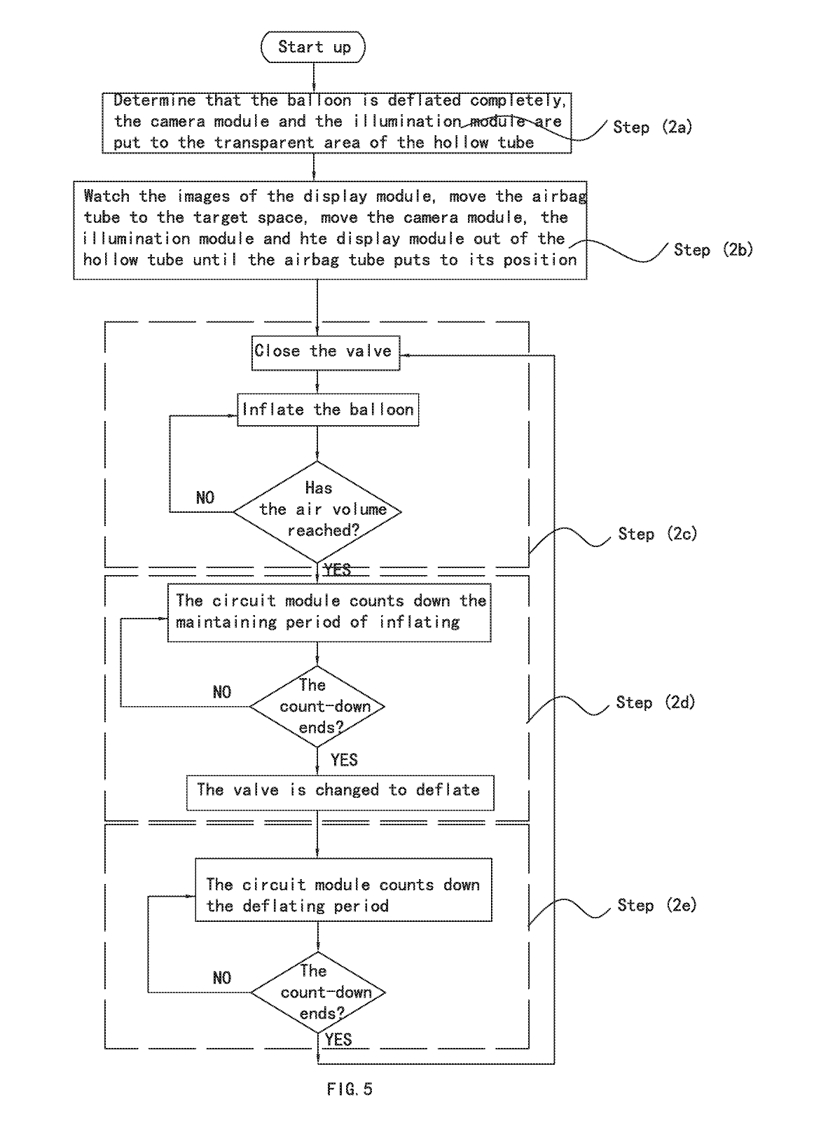

Referring to FIG. 4, an operating method of the airbag tube apparatus comprises the steps:

(1a) the operator determines that the balloon 12 is deflated completely, the balloon shrinks until the apparatus can enter the animal tissue;

(1b) the operator puts the airbag tube 10 to a target position, like rectum or urethra of a human body;

(1d) the circuit module 16 counts down (according to the preset inflating maintaining period), the valve 14 is changed at the end of the period, the gases in the balloon 12 leaks out to deflate, the balloon 12 shrinks that the apparatus can not flexibly fix to the animal tissue;

(1e) the circuit module 16 counts down according to preset deflating maintaining period, performing step (1c) at the end of the period, the balloon 12 is inflated, held and deflated automatically in accord with a preset period.

The present invention further comprises at least one balloon 12, at least one vent 13, at least one valve 14 and at last one side hole 8; the circuit module 16 controls the pneumatic module 15 and the valve 14 to inflate, hold and deflate the balloons 12. The balloons are changed to press the tissue area, preventing the problem of ischemic necrosis of the tissue like intestine wall due to overlong inflecting period of balloon.

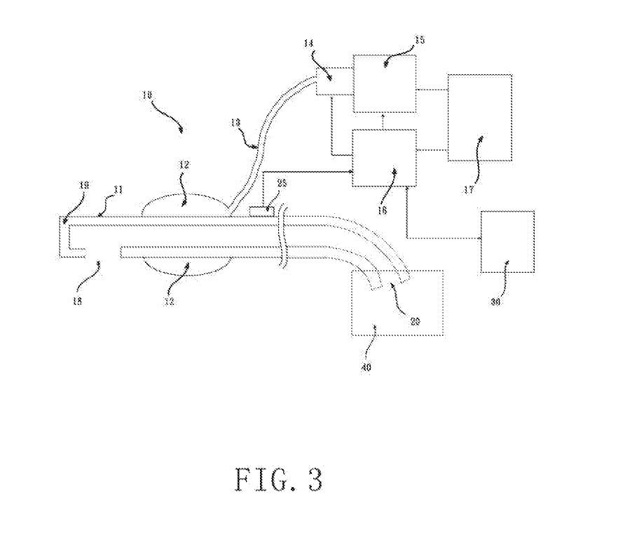

Referring to FIG. 2 and FIG. 3, the present invention is further provided that the hollow tube 11 is disposed with a transparent area, a camera module 22, an illumination module 21 and a display module 24; the camera module 22 comprises necessary power wires and signal wires. The camera module 22 moves in and out of the hollow tube 11; the transparent area of the hollow tube 11 is provided for the illumination module 21 to illuminate of and for the camera module 22 to shoot the periphery of the hollow tube 11; the transparent area of the hollow tube 11 can be a partial area or the whole area, for example, if the closed end 19 is transparent, images are transmitted to the display module 24, the operator can view the images from the display module 24, the display module 24 is a screen, a tablet computer, a computer or a hand phone; the illumination module 21 is a visual light resource, an infrared resource or any other light resource with any wavelength; the camera module 22 is configured with a panoramic lens, a non-panoramic lens, a wide-angle lens, a non-wide-angle lens, a fish-eye lens or other camera lens; the camera module 22 further comprises a ultrasonic transmitting and receiving unit. The operator can view the situations in the path of the apparatus in the animal body with the help of the illumination, the camera and the screen, preventing the apparatus piercing or damaging the tissue.

Referring to FIG. 5, the present invention further provides an operating method of the airbag tube apparatus with camera module, illumination module and display module, the method comprises the steps:

(2a) the operator determines that the balloon 12 is deflated completely, the camera module 22 and the illumination module 21 are put in the transparent area of the hollow tube 11, and the images of the camera module 22 displays in the display module 24;

(2b) the operator views the images of the display module 24, and moves the airbag tube 10 to the target area; after the position of the airbag tube 10 is determines, to prevent the apparatus piercing or damaging the tissue, the operator moves the camera module 22, the illumination module 21 and the display module 24 out of the hollow tube 11, making the camera module 22, the illumination module 21 and the display module 24 not contacted with the hollow tube 11. At this time, a drainage bag 40 can be provided to connect to the open end 20 of the hollow tube 11 for drainage;

(2c) the circuit module 16 controls the valve 14 to maintain the air of the balloon 12, the pneumatic module 15 inflates until the air volume of the balloon 12 reaches the preset parameter, the balloon has the volume increased to make the apparatus flexibly fix to the animal tissue.

(2d) the circuit module 16 counts down according to the maintain period; the circuit module 16 changes the valve 14 at the end of the maintain period and deflates the balloon 12; the balloon shrinks that the apparatus can not flexibly fix to the animal tissue;

(2e) the circuit module 16 counts down according to the deflation period; the circuit module 16 performs step (2c) at the end of the deflation period;

The operator can watch the video the camera module 22 films during the airbag tube 10 moves to the target area; the balloon 12 is inflated, held and deflated automatically in accord with a preset period. The operator can view the situations in the path of the apparatus in the animal body, preventing the apparatus piercing or damaging the tissue.

Referring to FIG. 3, the apparatus of the present invention further comprises a detecting module 25 attached to the hollow tube 11, the detecting module 25 is electrically connected to the circuit module 16 to provide detecting value, like resistance value, capacitance value, inducting value, PH, etc, to the circuit module 16. The circuit module 16 judges when there is a change and determines a preset situation, it sounds out or alarms to automatically detecting that whether the airbag tube slides out of the animal body.

Referring to FIG. 3, the present invention is further provided that the circuit module 16 is configured with communication function to communicate with a remote device 30 to provide the present information of the airbag tube 10 for long-distance operators or to receive parameters amended by long-distance operators through the remote device 30. The communication function is selected from WiFi, internet, Bluetooth, wire communication, wireless communication, optical communication;

the remote device 30 is selected from computer, hand phone and tablet computer. This provides long-distance operators more detail information and more convenient operation.

Each module and component have function to automatically maintain the air pressure of the balloon 12 according to the preset parameters, achieving automatically processing leaking problem of the balloon.

Referring to FIG. 1 and FIG. 2, the present invention further comprises a steering unit 23 of the camera module 22 with mechanism and function to control the direction of the camera module 22 and the closed end 19 of the hollow tube 11. This mechanism and function is similar to a controllable camera of endoscopic or the same at the front end of the endoscopic.

Please referring to FIG. 3, the present invention further comprises a drainage bag 40, the open end 20 of the hollow tube 11 is connected to the drainage bag 40, the drainage bag 40 is used to receive the drainage, the drainage bag 40 is configured with an outlet or without an outlet. When the apparatus is used to drain some matter, the drainage bag is used to store the matter like urine and faeces.

The method of the present invention further comprises a step that when the airbag tube 10 is put to the target area, the operator can operate the steering unit 23 of the camera module 22 to control the direction of the camera module 22 and the closed end 19 of the hollow tube 11. The operator can view the situations in the path of the apparatus in the animal body, preventing the apparatus piercing or damaging the tissue.

The valve 14 of the present invention is a check valve, which doesn't need the circuit module 16 to control its close.

Although the present invention has been described with reference to the preferred embodiments thereof for carrying out the patent for invention, it is apparent to those skilled in the art that a variety of modifications and changes may be made without departing from the scope of the patent for invention which is intended to be defined by the appended claims.

1. An airbag tube apparatus, comprising:
a hollow tube (11), a balloon (12), a vent (13), a valve (14), a pneumatic module (15), a circuit module (16), a power supply (17); wherein one end of the hollow tube (11) is a closed end (19), and the other end is the open end (20); a side hole (18) is disposed near the closed end (19), the balloon (12) is attached to the hollow tube (11) at a position far away from the side hole (18) of the closed end (19); the vent (13) connects the balloon (12), the valve (14) and the pneumatic module (15) to form an air inflator to inflate, hold and deflate the balloon; the circuit module (16) comprises a microprocessor unit to control the open and close of the valve (14) and the inflating of the pneumatic module (15), the power supply (17) supplies power to the apparatus; the hollow tube (11) is disposed with a transparent area, a camera module (22), an illumination module (21) and a display module (24); the camera module (22) moves in and out of the hollow tube (11); the transparent area of the hollow tube (11) is provided for the illumination module (21) to illuminate of and for the camera module (22) to shoot the periphery of the hollow tube (11); the pictures are transmitted to the display module (24), from which operators can watch the pictures; the operator can watch the video the camera module (22) films during the airbag tube (10) moves to the target area; the balloon (12) is inflated, held and deflated automatically in accord with a preset period.

2. The airbag tube apparatus according to claim 1, wherein the hollow tube (11) is disposed with X-ray imaging material; the apparatus comprises at least one balloon (12), at least one vent (13), at least one valve (14) and at last one side hole (8); the circuit module (16) controls the pneumatic module (15) and the valve (14) to inflate, hold and deflate the balloons (12).

3. The airbag tube apparatus according to claim 1, wherein the display module (24) is a screen, a tablet computer, a computer or a hand phone; the illumination module (21) is a visual light resource or an infrared resource; the camera module (22) is configured with a panoramic lens, a non-panoramic lens, a wide-angle lens, a non-wide-angle lens, a fish-eye lens; the camera module (22) further comprises a ultrasonic transmitting and receiving unit.

4. The airbag tube apparatus according to claim 1, wherein further comprising a detecting module (25) attached to the hollow tube (11), the detecting module (25) is electrically connected to the circuit module (16) to provide detecting value to the circuit module (16).

5. The airbag tube apparatus according to claim 1, wherein the circuit module (16) is configured with communication function to communicate with a remote device to provide the present information of the airbag tube (10) for long-distance operators or to receive parameters amended by long-distance operators through the remote device.

6. The airbag tube apparatus according to claim 5, wherein the communication function is selected from WiFi, internet, Bluetooth, wire communication, wireless communication, optical communication; the remote device (30) is selected from computer, hand phone and tablet computer.

7. The airbag tube apparatus according to claim 1, wherein each module and component have function to automatically maintain the air pressure of the balloon (12) according to the preset parameters.

8. The airbag tube apparatus according to claim 1, wherein further comprising a steering unit (23) of the camera module (22) with mechanism and function to control the direction of the camera module (22) and the closed end (19) of the hollow tube (11).

9. The airbag tube apparatus according to claim 1, wherein further comprising a drainage bag (40), the open end (20) of the hollow tube (11) is connected to the drainage bag (40), the drainage bag (40) is used to receive the drainage, the drainage bag (40) is configured with an outlet or without an outlet.

10. The airbag tube apparatus according to claim 1, wherein the valve (40) is a check valve, a bi-directional valve or a change valve, the valve (14) is controlled by the circuit module (16) or is not controlled by the circuit module (16).

11. An operating method of the airbag tube apparatus according to claim 1, wherein comprising the steps:
(2a) the operator determines that the balloon (12) is deflated completely, the camera module (22) and the illumination module (21) are put in the transparent area of the hollow tube (11), and the images of the camera module (22) displays in the display module (24);
(2b) the operator views the images of the display module (24), and moves the airbag tube (10) to the target area; after the position of the airbag tube (10) is determines, the operator moves the camera module (22), the illumination module (21) and the display module (24) out of the hollow tube (11), making the camera module (22), the illumination module (21) and the display module (24) not contacted with the hollow tube (11);
(2c) the circuit module (16) controls the valve (14) to maintain the air of the balloon (12), the pneumatic module (15) inflates until the air volume of the balloon (12) reaches the preset parameter;
(2d) the circuit module (16) counts down according to the maintain period; the circuit module (16) changes the valve (14) at the end of the maintain period and deflates the balloon (12);
(2e) the circuit module (16) counts down according to the deflation period; the circuit module (16) performs step (2c) at the end of the deflation period;
The operator can watch the video the camera module (22) films during the airbag tube (10) moves to the target area; the balloon (12) is inflated, held and deflated automatically in accord with a preset period.

12. The method according to claim 11, wherein the apparatus further comprises a steering unit (23) of the camera module (22) with mechanism and function to control the direction of the camera module (22) and the closed end (19) of the hollow tube (11); the method further comprises a step that when the airbag tube (10) is put to the target area, the operator can operate a steering unit (23) of the camera module (22) to control the direction of the camera module (22) and the closed end (19) of the hollow tube (11).
